(19) United States
(12) Patent Application Publication
(10) Pub. No.: US 2010/0150879 A1
(43) Pub. Date: Jun. 17, 2010

(54) Methods for the detection, isolation, and use of lipid phosphate phosphatase-3-positive stem cells

(76) Inventor: Kishore K. Wary, Elmhurst, IL (US)

Correspondence Address: Jane Massey Licata;Licata & Tyrrell P.C., 66 E. Main Street, Marlton, NJ, 08053, US

(21) Appl. No.: 12/579,837

(22) Filed: Oct. 15, 2009

Related U.S. Application Data
(60) Provisional application No. 61/106,019, filed on Oct. 16, 2008.

(51) Int. Cl. A61K 35/12; G01N 33/567; C12N 5/0789; C07K 16/00; A61P 17/02; A61P 35/00

(52) U.S. Cl. 424/93.7; 435/7.21; 435/325; 530/391.7; 435/375

(57) ABSTRACT

The present invention embraces antibodies that bind Lipid Phosphate Phosphatase-3, and use thereof in the detection and isolation of stems cells for promoting wound healing, neovascularization, and/or angiogenesis.

### INTRODUCTION

This application claims the benefit of priority from U.S. Provisional Application No. 61/106,019, filed Oct. 16, 2008, the content of which is incorporated herein by reference in its entirety.

This invention was made in the course of research sponsored by the National Institutes of Health, grant numbers HL079356. The U.S. government has certain rights in this invention.

### BACKGROUND OF THE INVENTION

Cell-cell and cell-matrix adhesion events regulate numerous complex biological processes including embryonic development, neovascularization, wound healing, and the formation of confluent endothelial cell (EC) monolayer (Adams & Watt (1993) Development 117:1183-1198; Bazzoni & Dejana (2004) Physiol. Rev. 84:869-90; Bohnsack & Hirschi (2004) Cell Cycle 3:1506-1511; Humtsoe, et al. (2003) EMBO J. 22:1539-1554; Risau (1997) Nature 386:671-674). However, the mechanism of formation of confluent EC monolayers required for the development of branching point structures is poorly understood (Carmeliet (2003) Nat. Med. 9:653-60; Davis & Senger (2005) Circ. Res. 97:1093-1107; Folkman & D'Amore (1996) Cell 87:1153-1155; Gory-Faure, et al. (1999) Development 126:2093-2102; Hoppler & Kavanagh (2007) J. Cell Sci. 120:385-393; Horowitz & Simons (2008) Circ. Res. 103:784-795; Kai, et al. (1996) J. Biol. Chem. 271:18931-18938; Prokhortchouk, et al. (2001) Genes & Dev. 15:1613-1618). Adherens junctions, which mediate cell-cell adhesion between ECs, may be involved in signaling EC confluence (Bazzoni & Dejana (2004) supra; Gory-Faure, et al. (1999) supra; Vestweber (2008) Thromb. Vasc. Biol. 28:223-232; Wallez & Huber (2008) Biochim. Biophys. Acta. 1778:794-809). VE-cadherin, a protein found in AJs, is a single-pass transmembrane polypeptide responsible for calcium-dependent homophilic interaction through its extracellular domains (Bazzoni & Dejana (2004) supra; Vestweber (2008) supra; Wallez & Huber (2008) supra). The VE-cadherin cytoplasmic domain interacts with the Armadillo domain-containing proteins, β-catenin, γ-catenin (plakoglobin), and p120-catenin (Bazzoni & Dejana (2004) supra; Grosheva, et al. (2001) J. Cell. Sci. 114:695-707; Vestweber (2008) supra; Wallez & Huber (2008) supra; Wildenberg, et al. (2006) Cell 127:1027-1039). Genetic and biochemical evidence shows the crucial role of β-catenin in regulating cell adhesion as well as proliferation secondary to β-catenins' central position in the Wnt signaling pathway (Goodwin & D'Amore (2002) Angiogenesis 5:1-9; Hoppler & Kavanagh (2007) supra; Korswagen & Clevers (1999) Cold Spring Harb. Symp. Quant. Biol. 64:141-147; Parmalee & Kitajewski (2008) Curr. Drug Targets 9:558-564; Zerlin, et al. (2008) Angiogenesis 11:63-69). In addition, the juxta-membrane protein p120-catenin (p120ctn) by binding to VE-cadherin also regulates AJ stability (Bazzoni & Dejana (2004) supra; Daniel & Reynolds (1999) Mol. Cell. Biol. 19:3614-3623; Davis, et al. (2003) J. Cell Biol. 163:525-534; Grosheva, et al. (2001) supra; Ichii & Takeichi (2007) Genes Cells 12:827-839; Noren, et al. (2000) J. Cell Biol. 150:567-580; Prokhortchouk, et al. (2001) supra; Wildenberg, et al. (2006) supra). Failure or inappropriate regulation of the β-catenin and VE-cadherin functions are linked to cardiovascular disease and tumor progression (Bazzoni & Dejana (2004) supra; Carmeliet (2003) supra).

Lipid Phosphate Phosphatase-3 (LPP3), also known as phosphatidic acid phosphatase-2b (PAP2b), was identified in a functional assay of angiogenesis (Humtsoe, et al. (2005) Biochem. Biophys. Res. Commun. 335:906-919; Humtsoe, et al. (2003) supra; Wary, et al. (2003) Mol. Cancer. 2:1-12; Wary & Humtsoe (2005) Cell Commun. Signal. 2:1-10). LPP3 not only has lipid phosphatase activity, but can also function as a cell-associated integrin ligand (Humtsoe, et al. (2005) supra; Humtsoe, et al. (2003) supra; Sciorra & Morris (2002) Biochim. Biophys. Acta 1582:45-51; Wary, et al. (2003) supra; Wary & Humtsoe (2005) supra). The family of known LPPs (LPP1, LPP2, LPP3) (Hynes (2007) J. Thromb. Haemost. 5:32-40; Ichii & Takeichi (2007) supra; Jia, et al. (2003) FEBS Lett. 552:240-246; Kai, et al. (1996) supra) are six transmembrane domain containing, plasma-membrane-bound enzymes that dephosphorylate sphingosine-1-phosphate (S1P) and its structural homologues, and thus generate lipid mediators (Brindley & Pilquil (2008) J. Lipid Res. doi:10.1194/jlr.R800055-JLR200; Burnett & Howard (2003) EMBO Rep. 4:793-799; Kai, et al. (1996) supra; Sciorra & Morris (2002) supra; Waggoner, et al. (1999) Biochim. Biophys. Acta. 439:299-316). All LPPs, which contain a single N-glycosylation and putative lipid phosphatase motif, are situated such that their N- and C-termini are within the cell (Brindley & Pilquil (2008) supra; Burnett & Howard (2003) supra; Jia, et al. (2003) supra; Kai, et al. (1996) supra; Sciorra & Morris (2002) supra; Waggoner, et al. (1999) supra). Only the LPP3 isoform contains an Arg-Gly-Asp (RGD) sequence in the second extracellular loop enabling it to bind integrins (Humtsoe, et al. (2005) supra; Humtsoe, et al. (2003) supra; Jia, et al. (2003) supra). Transfection experiments with green fluorescent protein (GFP)-tagged LPP1 and LPP3 showed that LPP1 is apically-sorted, whereas LPP3 co-localized with E-cadherin at cell-cell contact sites with other Madine Derby Canine Kidney (MDCK) cells (Jia, et al. (2003) supra). In addition, retroviral transduction of LPP3 induces cell aggregation/cell-cell interactions, modestly increases p120 catenin expression and promotes activation of the Fak, Akt and GSK3β protein kinases. Furthermore, expression of recombinant LPP3 promotes adhesion, spreading and tyrosine phosphorylation of Fak, Shc, Cas and paxillin in endothelial cells (Humtsoe, et al. (2003) supra).

Mutagenesis and domain swapping experiments established that the LPP1 contains an apical targeting signal sequence (FDKTRL; SEQ ID NO:1) in its N-terminal segment. In contrast, LPP3 contains a dityrosine (109Y/110Y) basolateral sorting motif (Jia, et al. (2003) supra). Notably, conventional deletion of Lpp3 is embryonic lethal, as the Lpp3 gene plays a critical role in extra-embryonic vasculogenesis independent of its lipid phosphatase activity (Escalante-Alcalde, et al. (2003) Development 130:4623-4637).

Rabbit anti-human LPP3 polyclonal antibodies to LPP3 have been described (Humtsoe, et al. (2003) supra; Wary, et al. (2003)Mol. Cancer. 2:1-12). In addition, a mouse anti-human LPP3 monoclonal antibody raised against the second extracellular loop of LPP3 is also effective in blocking experimental angiogenesis in vitro. Using such antibodies, it was demonstrated that LPP3 co-localizes with VEGF, αᵥβ₃ integrin, and von Willebrand Factor (vWF) in angiomas and is up-regulated in tumor endothelium (Wary & Humtsoe (2005) supra). Moreover, LPP3 has also been shown to play a role in EC adhesion, as a neutralizing antibody that binds to LPP3 was shown to prevent EC adhesion to other ECs (Humtsoe, et al. (2003) supra; Wary & Humtsoe (2005) supra) and prevent angiogenesis (Wary & Humtsoe (2005) supra).

### SUMMARY OF THE INVENTION

The present invention features methods for detecting and isolating stem cells from a sample using an anti-Lipid Phosphate Phosphatase 3 (LPP3) antibody. In some embodiments, the anti-LPP3 antibody is conjugated to a label or attached to a substrate. In other embodiments, the stem cells are pluripotent, multipotent, or progenitor stem cells. In particular embodiments, the stem cells are angioblasts or endothelial progenitor cells.

The present invention also features a method for promoting wound healing, neovascularization or angiogenesis. This method involves administering to a subject in need of treatment an effective amount of stem cells isolated via an anti-LPP3 antibody so that wound healing, neovascularization or angiogenesis is promoted.

A composition composed of an anti-LPP3 antibody directly or indirectly conjugated to a cancer cell killing agent and a method for using the same to kill cancer stem cells are also provided.

### BRIEF DESCRIPTION OF THE DRAWINGS

FIG. 1 depicts pLNCX2 retroviral constructs (a-h). The relative positions of RGD, RGE, RAD, phosphatase-defective (PD) mutations are shown. Murine Lpp3 (Construct-d) is the mouse counterpart to human LPP3. Numbers 1-6 represent transmembrane segments. The proposed cell binding sequence on the second extracellular loop of LPP3 is shown. Three copies of influenza virus derived hemagglutinin (HA) epitopes were fused N-terminal and in-frame to the open reading frame of the cDNAs.

FIG. 2 depicts a model of LPP3-regulated β-catenin signaling in subconfluent and confluent ECs. In sub-confluent ECs, LPP3 protects degradation of β-catenin in a PTEN-dependent manner. This event induces stabilization and translocation of β-catenin to the nucleus, where β-catenin interacts with LEF-1/TCF and displaces transcriptional repressors Groucho/transducin-like Enhancer of split (TLE) to activate transcription of Wnt target genes (e.g., fibronectin). In confluent ECs, excess β-catenin is phosphorylated, and the phosphorylated β-catenin is then subjected to proteosomal degradation. Consequently, p120ctn interacts with LPP3 at the plasma membrane, and contributes to the mechanism of EC-EC adhesion and adherens junction formation, thereby limiting the extent of EC migration.

### DETAILED DESCRIPTION OF THE INVENTION

It has now been found that anti-Lipid Phosphate Phosphatase 3 (LPP3) antibodies detect a subset of primitive stem and progenitor stem cells including angioblasts and endothelial progenitor cells (EPCs). Under normal physiological conditions, stem cells replace dead and aging tissues, contribute to wound healing, and promote neovascularization and angiogenesis. In cancer, stem cells can accelerate tumor progression and/or recurrence of cancer. Accordingly, the present invention embraces the detection and isolation of LPP3-positive stem cells for use in promoting neovascularization, e.g., in ischemic disease; promoting wound healing; promoting angiogenesis; and treating Acute Lung Injury (ALI) and traumatic brain injury (TBI). Moreover, given the role of cancer stem cells in cancer progression and recurrence, anti-LPP3 antibodies find use in the preparation of conjugates (e.g., an immunotoxin) for selectively targeting and killing LPP3-positive cancer stem cells.

For the purposes of the present invention, a stem cell is characterized by the ability to renew itself through mitotic cell division and differentiate into specialized cell types. Stem cells according to the invention include pluripotent, multipotent, or progenitor stem cells. In particular embodiments, the stem cell is an angioblast or endothelial progenitor cell. Cancer stem cells are also included within the scope of a stem cell. Cancer stem cells are cancer cells (found within tumors or hematological cancers) that possess characteristics associated with normal stem cells, specifically the ability to self-renew and give rise to all cell types found in a particular cancer sample. Such cells are proposed to persist in tumors as a distinct population and cause relapse giving rise to new tumors.

Thus, the present invention embraces a method for detecting stem cells by contacting a sample with an anti-LPP3 antibody and determining binding of the antibody to stem cells of the sample. As used herein, the term “sample” is meant to include a specimen or culture obtained from any source. Biological samples may be obtained from animals (including humans), e.g., by biopsy, and encompass fluids, solids, and tissues. Biological samples include blood products, such as plasma, serum and the like. A sample also includes a tissue biopsy suspected of being cancerous, wherein detection of the cancer stem cell is indicative or diagnostic of cancer or potential for cancer recurrence.

An anti-LPP3 antibody is used in the conventional sense to refer to an immunoglobulin, or fragment thereof, which specifically binds and recognizes an epitope present on the LPP3 protein. The LPP3 protein and its sequence are known in the art and provided under GENBANK Accession No. NP_{—}003704. In particular embodiments, the epitope is EGYIQNYRCRGDDSKVQEAR (SEQ ID NO:2), which includes the RGD motif. In other embodiments, the epitope is QNYKYDKAIVPESKNGGSPALNNNPRRSGSK (SEQ ID NO:3), which is the C-terminal domain of LPP3. In particular embodiments, the anti-LPP3 antibody does not recognize the RGD motif present in fibronectin nor does it recognize naked RGD peptide. An anti-LPP3 antibody of the invention also fails to recognize cryptic RGD sequence, such as RGD sequences that are exposed following proteolysis of collagen matrix proteins. In other words, the specificity of anti-LPP3 antibody is dictated by sequences flanking the RGD sequence3. In some embodiments, the anti-LPP3 antibody is monoclonal. In other embodiments, the anti-LPP3 antibody is polyclonal. In either case, the antibody can be produced using conventional methods. For instance, one can prepare polyclonal antibodies by immunizing animals such as rabbits with purified LPP3 protein or with peptides of a part thereof, collecting blood after a certain period of time, and removing blood clots. Further, one can prepare monoclonal antibodies by fusing antibody-producing cells of animals immunized with LPP3 protein, or peptides thereof, with bone tumor cells, isolating resultant single-clone cells (hybridoma) producing the objective antibodies, and obtaining antibodies from the cells. The antibodies thus obtained can be provided in a kit, panel or array and be used for the purification, detection and/or targeting of stem cells according to the present invention.

The form of the antibodies herein is not especially restricted, and, in addition to polyclonal antibodies and monoclonal antibodies, included are human antibodies, humanized antibodies made by gene recombination technique, low-molecular-weight antibodies, and, furthermore, their fragments and modified antibodies, as long as they bind to the LPP3-positive stem cells. In other words, the antibodies according to the present invention include polyclonal antibodies, monoclonal antibodies, chimeric antibodies, single-chain antibodies (scFv), humanized antibodies, and antibody fragments such as Fab, Fab′, F(ab′)₂, Fc, and Fv.

A variety of immunoassay formats may be used to select for anti-LPP3 antibodies and determine binding to LPP3 and/or LPP3-positive stem cells. For example, a conventional ELISA can be performed. To eliminate the need of secondary antibodies for detection, anti-LPP3 antibodies can be prepared as fusion proteins with β-galactosidase, maltose-binding protein, GST, green fluorescent protein (GFP), and such. Additional labels include biotin so that the antibody can be detected and/or collected with avidin or streptavidin. Moreover, immunofluorescence employs fluorescent labels, while other cytological techniques, such as histochemical, immunohistochemical and other microscopic (electron microscopy (EM), immunoEM) techniques use various other labels, either calorimetric or radioactive. The techniques may be carried out using, for example, anti-LPP3 antibodies conjugated with dyes, radioisotopes, or particles including magnetic beads. In this respect, the antibodies can be directly labeled and detected or alternatively detected indirectly, e.g., with a secondary antibody. Moreover, antibodies may be modified with PEG and such as needed.

The detection of LPP3-positive stem cells provides a basis for diagnosing the presence of cancer stem cells and also allows one to specifically differentiate between stem cells and other cells, thereby facilitating stem cell isolation. Accordingly, the present invention also embraces a method for isolating stem cells. In accordance with this method of the invention, a plurality of cells, preferably containing stem cells, is contacted with an anti-LPP3 antibody to bind the antibody to the stem cells, and the stem cells that are bound to the anti-LPP3 antibody are separated from the remaining cells of the plurality of cells. The plurality of cells according to this method are not especially restricted in terms of tissues and organs from which they are derived, and includes, for example, cell populations prepared (derived) from normal tissues and organs, cell populations prepared from morbid tissues, and cell populations prepared from cancer tissues. The cells may also be a mixture of cell populations composed of a plural species of cells prepared from various tissues, organs, and such.

Cells bound to the anti-LPP3 antibody can be separated by techniques that are well-known. For example, an anti-LPP3 antibody can be bound to a substrate, for instance the surface of a dish, filter (e.g., nitrocellulose filter or hollow fiber membrane), bead (e.g., agarose bead, polystyrene bead, or magnetic bead) or stent, wherein cells binding to the anti-LPP3 antibody adhere to the surface, while non-adherent cells can be washed off. Alternatively, the surface may be functionalized with an agent that binds an anti-LPP3 antibody; the cells of the sample are reacted with the anti-LPP3 antibody, and then subsequently the cells are reacted with the surface. The cells that bind to the anti-LPP3 antibody will therefore also adhere to the surface. This may be accomplished, for example, by using an anti-LPP3 antibody-biotin conjugate, and functionalizing the surface with streptavidin.

In methods based on fluorescence-activated cell-sorting, a plurality of cells is worked into a suspension and reacted with a fluorescent-tagged anti-LPP3 antibody. The cell suspension is entrained in the center of a stream of liquid. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescence of each cell is measured. An electrical charging ring is placed just at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge, thereby isolating the cells that are bound to the anti-LPP3 antibody. Desirably, stem cells isolated by the method of the invention are at least 70%, 80%, 90%, 95%, 99%, or 100% homogeneous to stem cells.

Stem cells isolated by the method of this invention can be used for a variety of applications including, but not limited to, research, drug screening and therapeutic applications such as promoting wound healing, neovascularization, or angiogenesis and treating Acute Lung Injury or traumatic brain injury. Thus, the present invention also embraces a method for promoting wound healing, neovascularization or angiogenesis by administering to a subject in need of treatment (e.g., those with a wound or condition wherein formation of functional microvascular networks, and protrusion and outgrowth of capillary buds and sprouts is desirable) stem cells isolated by the method of this invention. According to some embodiments, the stem cells are autologous or syngeneic, i.e., isolated from the subject being treated or from an identical individual. In other embodiments, the stem cells are allogeneic, i.e., isolated from another donor. In further embodiments, the stem cells can be undifferentiated at the time of administration, or alternatively cultured and differentiated into a particular cell type prior to administration. Subjects benefiting from treatment with stem cells of the invention will experience an alleviation, treatment or a delay of one or more signs or symptoms of the condition being treated as compared to a subject not receiving comparable treatment. As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

In addition to the isolation and detection of stem cells, the present invention also provides immunotherapies for selectively killing cancer stem cells. Accordingly, the present invention also embraces a composition composed of an anti-LPP3 antibody conjugated to a cancer cell killing agent and use thereof in a method for killing a cancer stem cell. For example, an anti-LPP3 antibody can be attached directly or indirectly to a cancer cell killing moiety, e.g., a therapeutic enzyme, cytotoxin (e.g., antimicrotubule derivative, DM1), cytokine (e.g., IL-2), radionuclide, anti-metabolite, or derivative thereof. Wherein the cancer cell killing agent is a protein, said agent can be expressed as a fusion protein with the anti-LPP3 antibody. Such recombinant protein technology is routinely practiced in the art using commercial recombinant expression systems. As indicated, the antibody can also be indirectly attached to the cancer cell killing agent, e.g., via a nanovector delivery system (e.g., cationic liposome, nanoparticle, or lipoplex), wherein the anti-LPP3 antibody facilitates entry of the cancer cell killing agent into cells. For example, the nanovector delivery system can be based upon the tumor-specific, ligand-targeting, self-assembled nanoparticle-DNA lipoplex system disclosed in U.S. Pat. No. 6,749,863. Such nanovector systems can employ a single chain antibody fragment (scFv) against a cell surface receptor which is overexpressed in the majority of human cancers.

As an alternative, the antibody can be used in pre-targeting therapies which in turn administer an antibody-(ligand or anti-ligand) conjugate which binds to targeted cells, followed by administration of a (ligand or anti-ligand) therapeutic moiety conjugate. Such methods are often favored over administration of antibody-therapeutic agent conjugates as they may reduce non-specific cytotoxicity. Also, it is desirable that the antibody be substantially non-immunogenic in the treated subject. This can be accomplished by chimerizing or humanizing the antibody, or producing a single chain version thereof. In addition, antibody fragments, such as Fab fragments are less immunogenic because of their smaller size. The killing of cancer stem cells is useful in the treatment of cancers and/or prevention of recurrence of cancers including, but not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

Methods for administering stem cells and antibodies are well-described in the art and include topical and parenteral modes of administration such as intramuscular and intravenous injection, as well as systemic routes of administration, e.g., oral, intranasal etc. Generally, a cell, antibody or antibody conjugate is administered in combination with a pharmaceutical carrier or excipient, and in conjunction with moieties that preserve the stability thereof, e.g., buffers. The dosage amount will vary within wide limits. For example, a suitable dose of antibody or antibody conjugate can vary from about 0.001 mg/Kg to 10 mg/Kg body weight.

As described herein, anti-LPP3 antibodies readily detect LPP3 thereby finding application in research, diagnosis and prognostic test kits in the detection and characterization of pluripotent, multipotent, and progenitor stem cells. In addition, anti-LPP3 antibodies can be used for drug screening using a cell-based assay and in small laboratory animals.

The invention is described in greater detail by the following non-limiting examples.

### Example 1

### Materials and Methods

Antibodies and Growth Factors. Preparation, purification, and characterization of rabbit anti-LPP3-RGD and anti-LPP3-C-cyto polyclonal antibodies have been described (Humtsoe, et al. (2005) supra; Humtsoe, et al. (2003) supra; Wary, et al. (2003) supra; Wary & Humtsoe (2005) supra). Mouse anti-VCIP (also called anti-LPP3) monoclonal, anti-LPP2 polyclonal antibody, and fluorescein isothiocyanate (FITC)-conjugated or TEXAS RED-conjugated goat or donkey IgGs were purchased from Invitrogen (Carlsbad, Calif.). A synthetic peptide (YRCRGDDSKVQEARKSFF; SEQ ID NO:4) was used to generate mouse anti-human LPP3 monoclonal antibody. Anti-phosphotyrosine-20 (PY20), anti-p120ctn (clone 98), anti-β-catenin (clone 14), and anti-γ-catenin (clone 15) monoclonal antibodies were purchased from BD Biosciences (San Jose, Calif.). The anti-human VE-cadherin (clone BV6) and anti-PTEN (Clone A2b1) monoclonal antibodies were purchased from Millipore/Chemicon International, Inc. (Temecula, Calif.). Mouse anti-human fibronectin (clone F-15), anti-hemaglutinin (HA) (clone 12CA5), anti-FLAG, and anti-pan-cadherin (CH-19) monoclonal antibodies were obtained from Sigma-Aldrich (St. Louis, Mo.). Rabbit anti-p-GSK3-β(S9) and anti-phospho-β-catenin (Ser33, Ser37, and Thr41) antibodies were purchased from Cell Signaling Technology, Inc. (Denver, Mass.). Rabbit Anti-p120 (S-19) polyclonal, anti-GST (Z-5), anti-Grb2, anti-LEF-1 (clone H-70) polyclonal and secondary antibodies were purchased from Santa Cruz Biotechnology (Santa Cruz, Calif.). Anti-CD31 (PECAM-1) (Clone 9G11) monoclonal antibody and recombinant human VEGF¹⁶⁵ and bFGF were purchased from R and D systems (Minneapolis, Minn.).

Recombinant cDNA and Short Hairpin (sh) RNA Constructs. The retroviral vector pLNCX2 and the amphotropic packaging cell line HEK293 (human kidney fibroblasts) were purchased from BD Biosciences. Wild-type human hLPP1, hLPP2, hLPP3, as well as mutant cDNAs were subcloned into the pLNCX2 retroviral vector directly downstream of the human CMV immediate early promoter. Two-step polymerase chain reaction (PCR) was used to insert three copies of the hemaglutinin (HA)-tag (YPYDVPDYA; SEQ ID NO:5) at the N-termini of all cDNAs as well as to generate an adhesion-defective (hLPP3-RAD), a phosphatase-defective (K(A)XXXXXXRP(A); SEQ ID NO:6) (hLPP3-PD), a double mutant (hLPP3-RAD+PD), and mutants lacking the N-terminal cytoplasmic domain (pLNCX2-HA-N-Δ-Cyto-hLPP3) or C-terminal cytoplasmic domain (pLNCX2-HA-C-Δ-Cyto-hLPP3) constructs (Humtsoe, et al. (2005) supra; Humtsoe, et al. (2003) supra). Newly generated constructs were sequence-verified. A set of four different shRNA constructs (in pLKO.1 lentiviral vector) targeting human LPP3, PTEN, and p120ctn genes were purchased from Sigma-Aldrich (St. Louis, Mo.). The retroviral or lentiviral particles were generated according to manufacturer's instructions. The efficiency of knockdown was determined by western analyses. FLAG-tagged PTEN wild-type and substrate-trapping mutant (C124S) cDNA constructs were a gift of Jack E Dixon (University of California, San Diego, Calif.). P120ctn cDNAs was obtained from Origene Technologies Inc. (Rockville, Md.) and [symbol] -catenin from Addgene (Cambridge, Mass.). One copy of HA-epitope was added in-frame to the C-terminus segments of these cDNAs by two-step PCR and sub-cloned into Sfi I site of pLNCX2 retrovirus as previously described (Humtsoe, et al. (2005) supra; Humtsoe, et al. (2003) supra). Correctness of sequences was verified by cDNA sequencing.

Cell culture, Retroviral Constructs, and Transfections. Human dermal microvascular endothelial cells (hdMVECs) were purchased from Lonza (Allendale, N.J.). HdMVEC cells were cultured according to conventional methods (Humtsoe, et al. (2003) supra; Wary, et al. (2003) supra; Wary & Humtsoe (2005) supra). In all assays, cells were grown in monolayers and used at either passage 3 or 4. Confluent ECs were split 1:4 or 1:1 so that on the day of experiments, the cells were approximately ˜50% or ˜100% confluent. Packaging and processing retroviral particles, and infection of targets cells were performed according to established methods (Humtsoe, et al. (2005) supra; Humtsoe, et al. (2003) supra). Because pooled populations of G418-resistant HEK293 cells were heterogeneous for LPP3 expression, cell clones were isolated and expanded. For transient transfection, cell culture supernatant containing retroviral particles was added to ECs plated at ˜40% density in presence of 8.0 μg/ml POLYBRENE and incubated for 12-16 hours. The following day, cells were cultured in complete media and incubated for 24-48 hours to allow maximal expression of recombinant proteins (Humtsoe, et al. (2005) supra; Humtsoe, et al. (2003) supra). Protein expression was analyzed by western blot.

Biochemical Experiments. For immunoprecipitation experiments, cells were rinsed with cold phosphate buffered saline (PBS), pH 7.4 and solubilized with complete cell lysis buffer (50 mM HEPES, pH 7.5; 150 mM NaCl; 1% TRITON X-100; 1 mM calcium chloride; 1 mM magnesium chloride; 1 mM sodium orthovanadate; 25 mM sodium fluoride; 1 mM sodium pyrosphosphate; 1 mM phenylmethylsulfonyl fluoride (PMSF); 10 μg/ml aprotinin; 10 μg/ml leupeptin; 1 μg/ml pepstatin-A) or with modified RIPA buffer (20 mM Tris, pH 7.5; 0.1% SDS; 0.25% sodium deoxycholate; 1% TRITON X-100; 150 mM sodium chloride; 1 mM sodium pyrosphosphate; 25 mM sodium fluoride; 1 mM sodium orthovanadate; 5 mM magnesium chloride; 1 mM calcium chloride; 1 μg/ml pepstatin; 1 mM phenylmethylsulfonyl fluoride; 1 μg/ml leupeptin) at 4° C. for 30 minutes. Cell extracts were centrifuged at ˜21,000×g for 30 minutes at 4° C., pre-adsorbed, and immunoprecipitated using specific antibodies. Immune complexes were washed 4-5 times with lysis buffer or with cold immunoprecipitation wash buffer (50 mM HEPES, pH 7.5; 150 mM NaCl; 0.5% NP-40; 1 mM calcium chloride; 1 mM magnesium chloride). SEPHAROSE-bound immune complexes were boiled in 1× Lammeli reducing sample buffer and resolved by SDS-PAGE. Western blot analysis was performed according to conventional methods (Humtsoe, et al. (2003) supra).

Far Western Analysis. For far western analyses, hdMVEC monolayers were solubilized in complete cell extraction buffer as described herein. Immunoprecipitation and western blot analyses were performed as described previously (Humtsoe, et al. (2005) supra; Humtsoe, et al. (2003) supra). The resulting blots were saturated with 5% milk in 1×TBS (25 mM Tris, 150 mM NaCl), pH 7.5 and then incubated for 1 hour with 2.0 μg/ml of soluble GST-LPP3-C-cyto fusion protein (bait) in TBS containing 0.1% TWEEN-20 and 2 mM dithiothreitol (DTT). After rinsing with TBS, membranes were analyzed by western blot with an anti-GST monoclonal antibody. For immunoprecipitation, antibodies were used at 2-5 μg per sample. For western blot, mouse monoclonal antibodies and rabbit polyclonal antibodies were used at a concentration of ˜0.5 μg/ml and ˜2.0 μg/ml, respectively.

Glutathione-S-Transferase (GST) Pull-Down Assay. The two-step PCR technique was used to generate in-frame fusions between GST and either the N-terminal cytoplasmic domain of LPP3 (GST-N-cyto or GST-NC) or the C-terminal cytoplasmic domain of LPP3 (GST-C-cyto or GST-CC). Two-step PCR, protein expression in E. coli, and the purification and elution of GST fusion proteins were performed as previously detailed (Humtsoe, et al. (2005) supra; Humtsoe, et al. (2003) supra). SDS-PAGE and COOMASSIE staining was performed to quantify purified GST-fusion protein and to verify the integrity of the protein.

Microscopy. For fluorescent microscopy, hdMVECs were grown on coverslips, fixed with 3% paraformaldehyde (PFA), and permeabilized with 0.5% TRITON X-100 in PBS, pH 7.4. Cells were subsequently incubated with the indicated primary antibodies, followed by the appropriate secondary donkey or goat anti-mouse IgGs conjugated either to FITC or TEXAS RED (Secondary antibody concentration, 2 μg/ml). PROLONG anti-fade (Invitrogen) containing DAPI was used to visualize nuclei. Microscopic analyses were performed using a ZEISS AXIOPLAN 2 microscope, and images were captured using a ZEISS digital camera equipped with AxioVision LE software.

Lymphoid Enhancer Binding Factor 1 (LEF-1) Luciferase Assay. For luciferase assays, constructs containing multimeric LEF-1 binding sites fused to a luciferase reporter gene (TOPFLASH) were used. hdMVEC cells were plated at three different densities (25-50%) in complete media so that after 48 hours the cells had reached 50%, 75%, and 100% confluence. These cells were then infected overnight (8 hours) with supernatants containing the indicated retroviral particles and allowed to recover for 4 hours in complete media. After recovery, the cells were co-transfected with TOPFLASH (3.0 μg/10⁶ cells) and a β-galactosidase (β-gal) normalization plasmid (0.5 μg/10⁶ cells) using SUPERFECT (QIAGEN Inc., Valencia, Calif.). After 4 hours of transfection, dishes were replenished with complete media. Luciferase and β-gal activities were assayed, at the indicated time, using the Luciferase Assay System (PROMEGA Corp., Madison, Wis.). Protein concentrations were adjusted for equivalent β-gal and luciferase activities. Experiments were repeated three times, using triplicate wells in each instance. In a subset of experiments, 25% confluent hdMVECs were infected for 12 hours with retroviral particles and allowed to recover in complete media for 6 hours prior to transient transfection. Transfection was performed, as described above, for 6 hours, and ECs were maintained in serum-free media (without growth factor) for an additional 12 hours prior to assay of β-gal and luciferase activity.

Enzyme-Linked ImmunoSorbent Assay (ELISA) Assay. ELISA assays were performed using commercially available kits (R & D systems, Biosource International, and Research Diagnostics). The limit of detection for each factor assayed ranged from 6 pg/ml (lower limit) to 1600 pg/ml (upper limit). The intra-assay variation was 4.3-6.5%, while inter-assay variation was approximately 6.5-10%.

Endothelial Cell Wound Healing Assay. Monolayer cultures of hdMVECs at 70% confluence (in 12-well plates) were infected overnight (˜16 hours) with either shLPP3 retroviral particles or retroviral particles carrying a control, non-silencing shRNA. Cultures were given fresh media and allowed to grow for 24 hours to form confluent monolayers. The monolayers were then scratched (wounded) gently with a 200 μl micropipette tip, washed twice in sterile PBS, and allowed to recover in defined media (EBM-2+0.1% BSA+1×ITS (insulin, transferrin, and selenium-A; Invitrogen)) at 37° C. At 0, 4, and 8 hours after injury, cultures were washed in PBS and fixed in 4% PFA in PBS (pH 7.5). Fixed cells were stained with eosin and hematoxylin, and images were obtained with a ZEISS phase-contrast microscope equipped with a digital camera.

Endothelial Cell Migration Assay. Cell migration assays were carried out using modified TRANSWELL Boyden (8 μm) chambers. Human dMVEC cells infected with shLPP3 or control shRNA retroviral particles were detached using 2 mM EDTA in PBS (pH 7.5). Cells were pelleted, washed once with PBS (pH 7.5), and resuspended in defined media. The upper chamber was filled with 500 μl of defined media containing ˜1×10⁴ cells and the lower chamber with 500 μl of defined media. VEGF (50 ng/ml), a chemoattractant, was added to the defined media in the lower chamber. Cells were incubated for 4 hours at 37° C. in a CO₂ incubator, and any cells that remained in the upper chamber after this incubation period were gently removed with Q-TIPS. Cells that had migrated to the lower side of the filter were fixed with 4% PFA, stained with 0.5% crystal violet, and washed to remove excess dye. The filter inserts were then mounted on glass slides and the cells were counted with a phase-contrast microscope. At least ten 200× magnification fields were randomly selected for each chamber-filter. Each experiment was repeated three times, with each trial being performed in triplicate.

Formation of Branching Point Structures. Morphogenic differentiation of ECs on type I collagen matrix was assessed by branching point structure formation as described with minor modifications (Humtsoe, et al. (2003) supra; Wary, et al. (2003) supra; Wary & Humtsoe (2005) supra). Briefly, ECs were detached from dishes with 3 mM EDTA, washed with PBS, suspended in defined medium (EBM-2 medium containing bFGF, VEGF¹⁶⁵, 1× insulin, transferrin, and selenium (ITS)), and plated onto type I collagen matrix. Approximately 0.1×10⁶ control ECs (at ˜50% density) or ECs were infected with either (1) control non-silencing shRNA (control sh), or (2) shRNA p120ctn (shp120ctn) (a), (3) shp120ctn (b), (4) shRNA LPP3 (shLPP3) (a), and (5) and shLPP3 (c) retroviral constructs for 8 hours, and fresh media added thereafter. After ˜16 hours, ECs were plated onto polymerized type I collagen gel and left at 37° C. for 12 hours. The unattached ECs were then removed, and a second layer of type I collagen gel was overlayed. After 36 hours, the media from dishes were removed, washed with PBS once and photographed under a phase-contrast microscope. For quantification of branching points, at least 10 random fields were chosen. Experiments were repeated three times.

Statistical Analysis. Comparisons of group means were performed using analysis of variance. Data were determined as mean±s.e.m. Values of p<0.05 were considered to be statistically significant.

### Example 2

### C-Terminal LPP3 Binding of p120ctn

ECs undergo cell-autonomous processes such as migration, proliferation, formation of confluent EC monolayer, and the formation of branching point structures, activities that are dependent on cell-cell and cell-matrix adhesion events. It has been shown that LPP3-induced cell-cell interactions are accompanied by increased expression of p120ctn (Humtsoe, et al. (2003) supra). Thus, it was determined whether cell density regulates LPP3 location and its function.

To study the interaction of LPP3 with p120ctn, hdMVECs were sparsely seeded and cultured for up to 96 hours in complete media containing bFGF and VEGF. Immunoprecipitation of lysates using anti-LPP3 antibody showed LPP3 associates with p120ctn in confluent ECs cells (i.e., the cells grown in high density for 72 and 96 hours), and not in sub-confluent cells. In a converse experiment, p120ctn-immunoprecipitate did not contain detectable amounts of LPP3 during the sub-confluent condition at 0, 24, or 48 hours of growth, whereas LPP3 was detected at 72 and 96 hours in the confluent ECs.

To address whether LPP3 binding to p120ctn requires VE-cadherin, cell extracts prepared from confluent ECs were subjected to immunoprecipitation with anti-VE-cadherin, anti-LPP3, and anti-p120ctn antibodies. Anti-VE-cadherin, anti-LPP3, and anti-p120ctn antibodies co-precipitated p120ctn. Anti-LPP3 and anti-p120ctn antibodies co-precipitated LPP3 in a reciprocal manner, whereas anti-VE-cadherin antibody did not. To exclude further the possibility that LPP3-p120ctn interaction was not mediated by VE-cadherin, cell extracts prepared from confluent ECs were subjected to immunodepletion with anti-VE-cadherin monoclonal antibody prior to immunoprecipitation with anti-LPP3, anti-p120ctn, or anti-mouse IgG (control) antibody. Anti-LPP3 and anti-p120ctn antibodies co-precipitated p120ctn and LPP3 in a reciprocal manner, whereas anti-mouse control IgG did not co-precipitate either LPP3 or p120ctn. These data show that interaction of LPP3 with p120ctn does not require VE-cadherin.

Subsequently, confocal imaging was used to examine distribution of LPP3 and p120ctn proteins 24- and 72-hour ECs using an anti-LPP3-RGD polyclonal antibody. At 24 hours, intense staining of p120ctn was observed in the nucleus; however, at 72 hours, when ECs were confluent, appreciable levels of p120ctn were recruited to the inner face at level of adherens junctions.

The LPP3-p120ctn interaction described above was confirmed in confluent ECs using ectopically-expressed LPP3 constructs. The construct encoding HA-tagged human wild-type LPP3 (pLNCX2-HA-WT-hLPP3) was stably transfected into HEK293 cells, which normally do not express LPP3. β-catenin, γ-catenin and p120ctn were not transfected into 293HEK cells, they were all endogenously expressed by these cells. Immunoprecipitation and western blot analyses of extracts prepared from HA-LPP3-expressing cells showed that HA-LPP3 co-immunoprecipitated with p120ctn. However, no HA-LPP3 was detected in β-catenin, γ-catenin, or mouse IgG immunoprecipitates. To determine whether HA-LPP3 was targeted to the HEK293 cell membrane, where it co-localizes with p120ctn, fluorescence labeling of HA and p120ctn was performed. In accord with the immunoprecipitation and western blot results above, HA-LPP3 was shown to co-localize with p120ctn.

To identify the region of LPP3 mediating the interaction with p120ctn, recombinant proteins were prepared by fusing GST to the N-terminal region (GST-N-cyto, QNYKYDKAIVPESKNGGSPALNNNPRRSGSK; SEQ ID NO:3) or C-terminal region (GST-C-cyto, SDLFKTKMTLSLPAPAIRKEILSPVDII DRNNHHNMM; SEQ ID NO:7) of LPP3. SDS-PAGE analysis showed that the proteins ran at the expected sizes with BSA (control) migrating at 68 kDa, GST alone at 29 kDa, GST-C-cyto (C-terminus fusion protein) at 32 kDa, and GST-N-cyto (N-terminus fusion protein) at 31 kDa. Far western analysis using GST fusion proteins as bait showed that GST-LPP3-C-cyto antibody bound to the 120 kDa p120ctn, but not to VE-cadherin, β-catenin, γ-catenin, or β₁-integrin. The identity of each protein, including p120ctn, was confirmed by re-probing membranes with antibodies against VE-cadherin, p120ctn, β-catenin, γ-catenin, and β₁-integrin. The VE-cadherin-associated p120ctn did not interact with recombinant LPP3. Pull-down assays were also carried out with GST-LPP3 fusion proteins. GST-C-cyto co-precipitated p120ctn. No p120ctn was detected in the GST-N-cyto precipitates. In these experiments, each of the membranes was probed for the presence of the GST-fusion protein. These data indicated that the C-terminal domain of LPP3 is required for binding with p120ctn and that the specific binding of LPP3 to p120ctn occurs only in confluent ECs.

### Example 3

### Loss of Endothelial Confluence Activates LPP3-Induced LEF-1 Transcriptional Activity

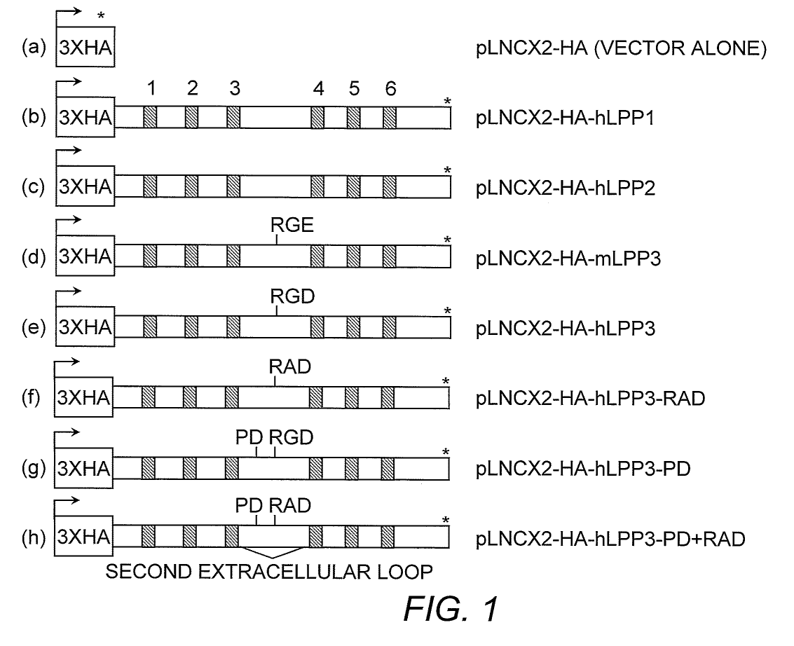

Initially, it was determined whether LPP3 induced the activation of LEF-1 using retrovirus-driven expression of wild-type LPP3 or LPP3 mutants (FIG. 1). The LEF-1-luciferase reporter assay was carried out in 50% confluent ECs to address the stimulatory effect of LPP3 in sub-confluent ECs. Transfection of empty vector (construct-a), hLPP1 (control construct-b), and hLPP2 (control construct-c) had no effect on luciferase activity; however, transfection of either mouse (m)Lpp3 (construct-d) or human (h)LPP3 (construct-e) induced 8- to 9-fold increase in luciferase activity.

Using LPP3-RAD mutant (construct defective in binding to integrin; construct-f, FIG. 1) and hLPP3-PD mutant (phosphatase-defective construct; construct-g, FIG. 1), the LPP3 domain responsible for the LEF-1 activity was subsequently identified. Transfection of construct-f and construct-g stimulated LEF-1-dependent transcription 3- and 5-fold, respectively, indicating that both domains activate LEF-1 activity. In contrast, the LPP3 mutant lacking both adhesion and lipid phosphatase domains (hLPP3-RAD+PD; Construct-h, FIG. 1) failed to stimulate luciferase activity.

To determine the mechanism of activation of LEF-1, ECs were transfected with constructs-a, or -b, or -c or -h (FIG. 1). No change in basal phosphorylation of β-catenin was observed using these constructs. However, the expression of mLpp3 (construct-d), hLPP3 (construct-e), hLPP3-RAD (construct-f), or hLPP3-PD (construct-g) in ECs decreased β-catenin phosphorylation, indicating that expression of these LPP3 constructs induced the stabilization of β-catenin. Notably, an interaction between β-catenin and LEF-1 was observed only in ECs expressing constructs-d, -e, -f, or -g. Thus, LPP3 functioned by preventing phosphorylation of β-catenin and in turn increased β-catenin stability, and binding of β-catenin to LEF-1 required for LEF-1/TCF transcriptional activity (Marsden & DeSimone (2001) Development 128:3635-3647; Noren, et al. (2000) supra).

To further analyze the stimulation of LEF-1 activation by LPP3, the expression of the specific LEF-1 target gene, fibronectin (Olsson, et al. (2006) Nat. Rev. Mol. Cell. Biol. 7:359-371; Park, et al. (2006) Dev. Cell 11:683-695), was determined. Expression of fibronectin is known to mediate EC adhesion, migration, proliferation, and cellular polarity (Olsson, et al. (2006) supra; Park, et al. (2006) supra; Parmalee & Kitajewski (2008) supra; Prokhortchouk, et al. (2001) supra; Risau (1997) supra). Thus, the production of fibronectin as well as a non-LEF-1-regulated protein, tissue inhibitor of metalloproteinases-2 (TIMP-2) was assayed in supernatants of 50% sub-confluent EC cultures using ELISA. This analysis indicated that fibronectin production in sub-confluent ECs was unaffected by transfection of constructs-a, -b, or -c or -h. In contrast, expression of construct-d or construct-e increased fibronectin concentrations by 5-fold. In addition, fibronectin secretion in ECs transfected with construct-f or construct-g was observed. The amount of TIMP-2 was unaltered following transfection of these constructs. Thus, LPP3 in sub-confluent ECs activates LEF-1-dependent transcription secondary to stabilization of β-catenin resulting in fibronectin production.

It was subsequently determined whether the transcriptional activity of LEF-1 was regulated by LPP3-mediated nuclear accumulation of dephosphorylated β-catenin. Based on the evidence that Phosphatase and TENsin homolog (PTEN), the dual-specificity phosphatase regulates phosphorylation of β-catenin (Sciorra & Morris (2002) supra; Sharma, et al. (2002) J. Biol. Chem. 277:30935-30941), the possible role of PTEN in LPP3-induced stabilization of β-catenin was determined. It was initially determined whether PTEN binds to β-catenin in sub-confluent ECs. Subsequently, a FLAG-tagged-wild-type PTEN construct or FLAG-tagged substrate-trapping PTEN mutant construct (catalytically inactive, PTEN-C124S) was transfected in sub-confluent ECs. Wild-type PTEN did not co-immunoprecipitate β-catenin; however, co-immunoprecipitation of β-catenin was observed with the phosphatase-defective (substrate trapping) PTEN-C124S. To determine whether PTEN stimulates dephosphorylation of β-catenin, and thereby increases its stability, sub-confluent ECs were transfected with vector alone (control), or phosphatase-defective PTEN-C124S, or with increasing concentration of wild-type PTEN, and β-catenin phosphorylation was examined. It was observed that transfection of PTEN resulted in decreased phosphorylation of β-catenin accompanied by increased nuclear accumulation of β-catenin in sub-confluent ECs without a change in total cellular β-catenin amount. This response did not occur following expression of phosphatase-defective PTEN-C124S. Thus, PTEN in sub-confluent ECs interacted with and stimulated dephosphorylation of β-catenin. Since dephosphorylated β-catenin is stable and translocates to the nucleus to activate LEF-1, the data indicate a critical role for PTEN in the mechanism of activation of β-catenin in sub-confluent ECs.

### Example 4

### PTEN Knockdown Affects LPP3 Mediated β-Catenin/LEF-1 Signaling

Sub-confluent ECs were used to examine the impact of PTEN knockdown on LPP3 mediated β-catenin/LEF-1 signaling. Cells were plated (0 hour); infected with shRNA particles at 16 hours after plating; transfected with HA-WT-hLPP3, TOPFLASH, and WT-PTEN constructs at 24 hours after plating; and LEF-1 luciferase assays were conducted at 36 hours after plating. In control sub-confluent ECs, the fold LEF-1 luciferase activity was basal, while transfection of LPP3 in these cells resulted in a 12-fold activation of luciferase. In contrast, PTEN-knockdown reduced LPP3-mediated LEF-1 activation down to 3-fold over basal, which was also reflected in increased phosphorylation of β-catenin. Decrease in LEF-1 activity in PTEN-knockdown ECs was restored, however, by ectopic expression of wild-type PTEN, and this effect was accompanied by increased β-catenin stability. The efficiency of knockdown, phosphorylation of β-catenin, and expression of LPP3 and PTEN were determined by western blot analysis.

### Example 5

### Re-Expression of β-Catenin Reverses the Effect of Loss of LPP3

The effects of shRNA-mediated silencing of LPP3 gene on the formation of EC branching point structures, an in vitro correlate of angiogenesis (Davis & Senger (2005) supra; Horowitz & Simons (2008) supra; Kamei, et al. (2006) Nature 442:453-456; Wary, et al. (2003) supra; Wary & Humtsoe (2005) supra) were analyzed. ECs were left untreated (control ECs), or transduced with control shRNA, or shLPP3(c) constructs. To examine whether β-catenin or p120ctn can reverse the loss of LPP3, LPP3 depleted ECs were either infected with retrovirus encoding full-length human β-catenin or full-length human p120ctn cDNAs. Transduced cells were then subjected to the branching point structure assay using three-dimensional type I collagen as the supporting matrix protein. For this assay, defined media containing bFGF, VEGF and ITS, but no serum was used. The branching point structures in control ECs were similar to ECs transduced with non-silencing control shRNA retroviral constructs. A significant decrease in branching point was observed in LPP3-depleted ECs. ECs infected with full-length p120ctn did not reverse the loss of LPP3. Notably, the formation of branching point structures was restored in ECs infected with retrovirus encoding full-length β-catenin. Thus, the loss of LPP3 can be reversed by expressing β-catenin.

To confirm whether expression of β-catenin rescues the phenotype caused by the loss of LPP3, LPP3 knockdown ECs were used, wherein p120ctn or β-catenin was ectopically re-expressed. On day 1 of this experiment, ECs were seeded at low density. On day 2, cells were either left untreated or subjected to shRNA-mediated LPP3 knockdown for 8 hours. On day 3, ECs at 60% density were either left alone or transfected with vector alone or HA-tagged-p120ctn or -β-catenin. After an 8-hour transfection, ECs were allowed to recover overnight in complete media. On day 4, cells were either plated onto coverslips for staining or solubilized in cell extraction buffer for western analysis. The results of this analysis indicated that LPP3 remained diffusely distributed in control ECs. Following LPP3 knockdown, expression of LPP3, β-catenin, VE-cadherin, and p120ctn proteins were reduced. Staining of ECs with anti-HA or anti-VE-cadherin showed that re-expression of p120ctn failed to induce confluent monolayer or defined adherens junction adhesion structures. Instead, these cells were elongated. Re-expression of β-catenin induced the formation of well-defined adherens junctions as determined by the intense p120ctn staining. Re-expression of β-catenin appeared to have induced expression of p120ctn, VE-cadherin, and fibronectin, indicating that β-catenin re-expression reverses alterations caused by the loss of LPP3. Alternatively, the results could be secondary effects due to protein stabilization by junction assembly.

### Example 6

### LPP3 is Required for Synthesis of Fibronectin and EC Migration

The effects of shRNA-mediated LPP3 gene silencing on fibronectin synthesis known to be regulated by LEF-1 (Loscertales, et al. (2008) Development 135:1365-1376; Marsden & DeSimone (2001) supra) was examined. Four shRNA constructs were used for these experiments: shRNA (negative control), shLPP3(a), shLPP3(b), and shLPP3(c) (FIG. 1). Constructs shLPP3(a), shLPP3(b), and shLPP3(c) were used because they target three distinct regions of human LPP3 gene. The efficacy of each construct, tested in mouse NIH-3T3 fibroblasts, showed no evidence of toxicity. In addition, no changes in morphology of ECs were observed. Expression of shRNA or shLPP3(a) did not affect the expression or localization of p120ctn, fibronectin, or the non-LEF-1-regulated control protein CD31 (PECAM-1). In contrast, expression of shLPP3(b) and shLPP3(c) decreased the synthesis of fibronectin without affecting expression of CD31. Knockdown of LPP3 was confirmed by western blot analysis.

Because fibronectin is a downstream target of Wnt-mediated β-catenin signaling (Loscertales, et al. (2008) supra; Marsden & DeSimone (2001) supra), analysis of β-catenin phosphorylation showed that increased phosphorylation was accompanied by decreased β-catenin expression. Moreover, phosphorylation of Ser-9 of GSK-3β, the kinase that in turn phosphorylates β-catenin, was observed. Phosphorylated β-catenin is usually proteolysed in the cytoplasm, a mechanism which limits the extent of β-catenin signaling.

To determine whether reduced LPP3 expression had a functional effect on EC migration, a wound healing assay using ECs infected with non-silencing control or shLPP3 was employed. The re-population of the wounded area was monitored at 0, 4, and 8 hours after scratching the monolayer. ECs expressing non-silencing control shRNA migrated through the wound region within 8 hours. In contrast, ECs expressing shLPP3(c) showed no cell migration. Subsequently, the TRANSWELL Boyden chamber cell migration assay was used following LPP3 knockdown. This analysis indicated that, in the absence of VEGF, cells receiving control shRNA showed 4% migration compared with <1% migration of cells expressing shLPP3 (p<0.05), whereas in the presence of VEGF, control cells showed 12% migration within 8 hours compared with 4% migration for ECs expressing shLPP3 (p<0.05). Thus, knockdown of LPP3 also impaired the ability of ECs to migrate in this assay system.

To address if impaired cell migration was due to reduced fibronectin synthesis, increasing concentrations of fibronectin (1.25, 2.4, 5.0, and 7.5 μg/ml) were added and wound closure and cell migration were determined. Maximum induction of migration was seen at 5.0 μg/ml fibronectin. Thus, these results collectively show that shRNA-mediated loss of LPP3 decreases fibronectin synthesis, and thereby impairs both EC migration and wound healing.

### Example 7

### C-Terminal Domain of LPP3 Regulates p120ctn and VE-Cadherin Expression and Formation of Branching Point Structures

To examine whether cell surface-associated LPP3 interacts with p120ctn, ECs transiently expressing pLNCX2-HA-WT-hLPP3, or the mutant constructs lacking the N-terminal cytoplasmic domain (SEQ ID NO:3; pLNCX2-HA-N-Δ-cyto-hLPP3) or C-terminal cytoplasmic domain (SEQ ID NO:7; pLNCX2-HA-C-Δ-cyto-hLPP3) were used. Cell surface biotinylation and co-immunoprecipitation were employed to monitor cell surface-associated interactions. The anti-HA antibody co-precipitated p120ctn in cells expressing HA-WT-hLPP3 and HA-N-Δ-cyto-hLPP3, but not HA-C-Δ-cyto-hLPP3. Wild-type and mutant-LPP3 produced smears in the blots in response to streptavidin-HRP reaction, indicating that LPP3 was exported to the cell surface. These smears were likely due to N-glycosylation of LPP3 (Wary, et al. (2003) supra). Although HA-C-Δ-cyto-hLPP3 was efficiently exported to the cell surface, it failed to co-localize with p120ctn. Thus, these data demonstrate that cell surface-associated LPP3 interacts with p120ctn and the C-terminus of LPP3 is required for this interaction.

To examine the function of LPP3-p120ctn interaction, endogenous LPP3 was deleted in ECs using shLPP3(c). ECs were then infected with HA-WT-hLPP3, HA-N-Δ-cyto-hLPP3, or HA-C-Δ-cyto-hLPP3 constructs and subjected to branching point, cell proliferation, and migration assays. Silencing with shLPP3(c) decreased the number of branching points to 4%. Re-expression of HA-WT-hLPP3 and HA-N-Δ-cyto-hLPP3 restored this value to 17% and 14%, respectively. In contrast, re-expression of HA-C-Δ-cyto-hLPP3 induced a minimal increase in branching points (7%). In addition, re-expression of HA-WT-hLPP3, HA-N-Δ-cyto-hLPP3, and HA-N-Δ-cyto-hLPP3 all caused an increase in both cell proliferation and cell migration.

Total cell extracts were prepared and p120ctn and VE-cadherin expression was measured. Increased expression of p120ctn and VE-cadherin was measured in response to re-expression of HA-WT-hLPP3 and HA-N-Δ-cyto-hLPP3, but not HA-C-Δ-cyto-hLPP3. Grb-2 protein expression was used as a control.

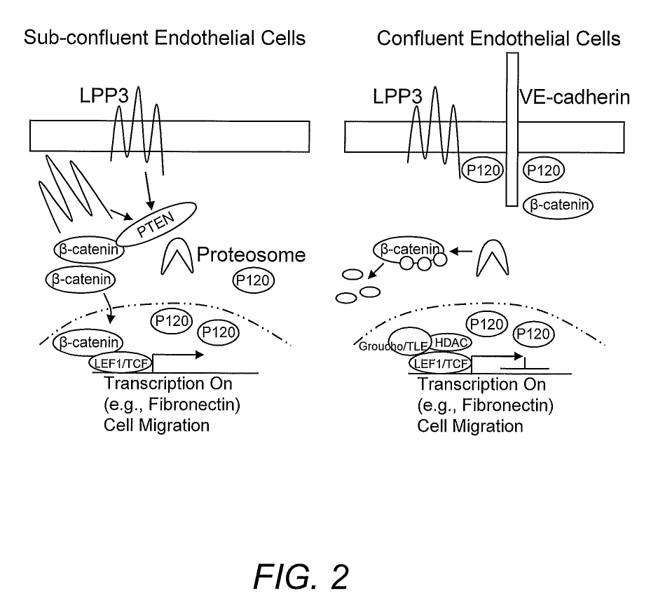

Thus, the data herein indicate that the C-terminal segment of LPP3 is required for p120ctn expression and regulation of p120ctn function, e.g., the maintenance of adherens junction integrity. When adherens junction integrity is lost, ECs display deregulated branching point structures due to destabilization of VE-cadherin from the cell surface, but gain a modest proliferative and migratory phenotype (FIG. 2).

What is claimed is:

1. A method for detecting stem cells comprising contacting a sample with an anti-Lipid Phosphate Phosphatase 3 antibody and determining binding of the antibody to stem cells of the sample so that stem cells in the sample are detected.

2. The method of claim 1, wherein the anti-Lipid Phosphate Phosphatase-3 antibody is conjugated to a label.

3. The method of claim 1, wherein the stem cells are primitive, pluripotent, multipotent, or progenitor stem cells.

4. The method of claim 3, wherein the primitive stem cells are angioblasts or endothelial progenitor cells.

5. A method for isolating stem cells comprising contacting a plurality of cells comprising stem cells with an anti-Lipid Phosphate Phosphatase-3 antibody; and separating the antibody-bound stem cells from remaining cells of the plurality of cells so that the stem cells are isolated.

6. The method of claim 5, wherein the anti-Lipid Phosphate Phosphatase 3 antibody is attached to a substrate, and the step of separating comprises removing the substrate away from the plurality of cells.

7. The method of claim 5, wherein the stem cells are primitive, pluripotent, multipotent, or progenitor stem cells.

8. The method of claim 7, wherein the primitive stem cells are angioblasts or endothelial progenitor cells.

9. Stem cells isolated by the method of claim 5.

10. A method for promoting wound healing, neovascularization or angiogenesis comprising administering to a subject in need of treatment the stem cells of claim 9 so that wound healing, neovascularization or angiogenesis is promoted.

11. A composition comprising an anti-Lipid Phosphate Phosphatase-3 antibody conjugated to a cancer cell killing agent.

12. The composition of claim 11, wherein the cancer cell killing agent is directly or indirectly conjugated to the anti-Lipid Phosphate Phosphatase-3 antibody.

13. A method for killing a cancer stem cell comprising contacting a cancer stem cell with the composition of claim 11 so that the cancer stem cell is killed.


SEQUENCE LISTING (7 sequences)

SEQ ID NO: 1 - protein, 6 aa, Artificial Sequence
  Synthetic peptide
FDKTRL

SEQ ID NO: 2 - protein, 20 aa, Artificial Sequence
  Synthetic peptide
EGYIQNYRCRGDDSKVQEAR

SEQ ID NO: 3 - protein, 31 aa, Artificial Sequence
  Synthetic peptide
QNYKYDKAIVPESKNGGSPALNNNPRRSGSK

SEQ ID NO: 4 - protein, 18 aa, Artificial Sequence
  Synthetic peptide
YRCRGDDSKVQEARKSFF

SEQ ID NO: 5 - protein, 9 aa, Artificial Sequence
  Synthetic peptide
YPYDVPDYA

SEQ ID NO: 6 - protein, 9 aa, Artificial Sequence
  Synthetic peptide
XXXXXXXRX

SEQ ID NO: 7 - protein, 37 aa, Artificial Sequence
  Synthetic peptide
SDLFKTKMTLSLPAPAIRKEILSPVDIIDRNNHHNMM